(19) United States
(12) Patent Application Publication
(10) Pub. No.: US 2014/0296749 A1
(43) Pub. Date: Oct. 2, 2014

(54) Body Monitoring System and Method

(71) Applicants: Lawrence G. Reid, JR., Germanton, NC (US); George T. Hicks, Walnut Cove, NC (US); Edward Grant, Raleigh, NC (US)

(72) Inventors: Lawrence G. Reid, JR., Germanton, NC (US); George T. Hicks, Walnut Cove, NC (US); Edward Grant, Raleigh, NC (US)

(21) Appl. No.: 14/225,952

(22) Filed: Mar. 26, 2014

Related U.S. Application Data
(60) Provisional application No. 61/805,175, filed on Mar. 26, 2013.

(51) Int. Cl. A61B 5/00; A61H 1/00

(52) U.S. Cl. CPC A61B 5/6804; A61B 5/0053; A61B 5/4836; A61H 1/006; A61B 5/6812
USPC 600/587; 600/300

(57) ABSTRACT

A body monitoring system can include a wearable device, and a circuit for conducting electrical signals having an electrically conductive yarn knitted into the device. The circuit can further include a sensor circuit configured to sense a variable in an area of a body to which the device is applied. The circuit can further include a transmission circuit configured to transmit an electrical signal representing a value of a variable in an area of a body to another location. The other location can be an external device separate from the wearable device, such as an electronic display unit.

### CROSS-REFERENCE TO RELATED APPLICATION

This application claims benefit of U.S. Provisional Patent App. No. 61/805,175, filed Mar. 26, 2013, which application is incorporated by reference herein in its entirety.

### FIELD OF THE INVENTION

The present invention relates to a body monitoring system and/or method.

### BACKGROUND OF THE INVENTION

Compressive pressure is utilized in the treatment and/or prevention of wounds, peripheral vascular disease, or other conditions. For such treatment or prevention to be effective, the amount of compressive pressure applied often must be accurate. Insufficient compression may result in suboptimal treatment. Excessive compression can retard blood flow, leading to detrimental results. In conventional compression wraps and garments, the actual amount of compressive force provided by the wrap/garment at its interface with an anatomical area when worn is unknown. Thus, without knowing the actual applied compressive pressure, the opportunity for effective clinical management of compressive therapy is diminished.

An amount of compressive pressure that a garment is capable of providing when applied to a patient can be determined prior to use. Compression fabrics/garments can be tested under stretch conditions and certified for compression ranges within a defined circumference fitting range. The amount of compression that a fabric or garment is capable of generating can be affected by various yarn and construction factors. Such factors can include, for example, yarn type and size (for example, denier); characteristics of elastic yarns utilized (for example, how an elastic yarn is extruded and/or wrapped, such as under how much tension); and fabric structure (for example, stitch pattern, size, and/or density).

However, applying accurate compressive pressure to a body with wraps/garments utilizing stretch fabrics poses numerous challenges. The actual amount of compressive pressure applied by a particular wrap/garment depends on various factors, including, for example, the number of fabric layers applied, the type of stretch material in each layer, the combined stretch characteristics of multiple layers and/or materials, body shape and circumference, and other variables. Thus, to provide optimal, or even clinically effective, compressive pressure therapy, the actual amount of compressive pressure being applied to a patient may need to be accurately measured.

Some conventional compression bandages have a design knitted into the fabric to indicate the amount of stretch that should be applied. For example, a compression garment can include a rectangular design, such that when the garment is stretched in one direction (for example, longitudinally) to a desired degree, the rectangle forms a square. Maintaining the square shape indicates that a particular amount of stretch is being maintained while the compression bandage is applied. However, an amount of stretch may not correlate with a particular amount of compressive pressure. That is, while such products may provide qualitative indications of compressive pressure, they do not measure the actual compressive force being applied.

In certain clinical situations, the compressive pressure along an anatomical area, such as a leg, needs to be graduated. Thus, it is important to know with accuracy the amount of compressive pressure being applied at various locations along the anatomical area. Using conventional compression garments without verification of actual applied compressive pressure(s) limit the ability to accurately apply graduated compressive pressure.

Another clinical situation in which it is important to know the actual compressive pressure being applied is when the patient has a reduction in edema underneath the compression garment. If the reduction in edema is sufficient to affect the amount of compressive pressure being applied, a smaller compression garment, a garment that provides a higher amount of pressure, or an additional compressive pressure layer may need to be applied. As an example, some compressive pressure systems apply compression with a high stiffness, or rigidity, factor. With a reduction in edema, a rigid compression system becomes unable to provide compression as the underlying anatomical area reduces in diameter and pulls away from the compression system. In this instance, knowing the actual compressive pressure being applied provides the information necessary to determine whether the compression system may need to be replaced in order for therapy to be continued.

Another disadvantage of conventional compression fabrics and garments is that the initial compressive force of such a garment when applied can often diminish over time as a consequence of yarn fatigue. Yarn fatigue can be defined as the weakening of a yarn caused by a loss of some of its ability to recover to its original shape or size after being deformed repeatedly. As a compression garment over time loses elasticity and the ability to provide the compressive force for which it was initially rated, it becomes important to know the actual amount of compressive pressure the garment provides after repeated and/or prolonged use.

Determining the compressive force between a person's body and a compressive pressure device can be determined utilizing a force or pressure gauge placed between the body and the compressive device. However, utilizing force gauges in this manner has limitations. Force gauges register only point pressure, which can vary depending on the radius of anatomical curvature at the point of measurement. Force gauge measurements of compressive pressure are also subject to variations related to the amount of body rigidity at the point of measurement. In addition, because force gauges are generally not incorporated into an entire compression garment, accurate measurement of an average compressive force in a particular area of the garment, or in an entire compressive garment, are not available.

Thus, there is a need for a means for easily and accurately determining an actual amount of compressive pressure applied to an anatomical area by a compressive pressure garment. There is a need for such a means for easily and accurately determining an actual amount of applied compressive pressure the entire time the garment is being worn. There is a need for such a means for easily and accurately determining an actual amount of applied compressive pressure regardless of variables related to yarn, fabric construction, stretch characteristics, number of fabric layers, yarn/fabric fatigue, body shape and circumference, etc.

### SUMMARY OF THE INVENTION

Some embodiments of the present invention include a body monitoring system comprising a wearable device, and a circuit for conducting electrical signals comprising an electrically conductive yarn knitted into the device. In some embodiments, the circuit can further comprise a sensor circuit configured to sense a variable in an area of a body to which the device is applied. In some embodiments, the circuit can further comprise a transmission circuit configured to transmit an electrical signal representing a value of a variable in an area of a body to another location. The sensor circuit can further comprise an electrical sensitivity for reliably sensing the variable. The transmission circuit can further comprise an electrical sensitivity for reliably transmitting the value of a variable.

In some embodiments, the electrically conductive yarn can comprise a silver yarn or a yarn coated with silver. For example, the electrically conductive yarn can be a single 70 denier silver yarn or two 70 denier silver yarns twisted together. In embodiments in which the electrically conductive yarn comprises stitch loops, the stitch loops are preferably packed together during knitting so that the stitch loops in adjacent courses along a particular wale have sufficient contact to provide a continuous circuit. In embodiments in which the electrically conductive yarn comprises nylon yarn having silver or a silver composition applied thereto, the nylon yarn can be heated sufficiently to shrink the nylon yarn so that stitch loops in adjacent courses along a particular wale have sufficient contact to provide a continuous circuit.

In various embodiments, the circuit can further comprise the electrically conductive yarn knit in a vertical, horizontal, or angled direction in the fabric. In one embodiment, the electrically conductive yarn comprises a knit rib pattern to provide a vertical circuit direction in the fabric. In another embodiment, the electrically conductive yarn is knit along a course to provide a horizontal circuit direction in the fabric. To provide an angled circuit direction in the fabric, the electrically conductive yarn can be knit in a wale offset from a previous wale in successive courses. In yet other embodiments, the electrically conductive yarn can be laid in: a single course to provide a horizontal circuit direction; in a plurality of courses to provide an angled circuit direction; or in changing directions between courses to provide a multi-directional circuit direction.

In some embodiments, the wearable device can comprise an elastic fabric having an unstretched dimension in a direction of the circuit. In such an embodiment, stretch beyond the unstretched dimension in the circuit direction can be limited to provide sufficient circuit continuity for reliable conduction of the electrical signals. For example, when the circuit comprises a cut yarn, stretch is limited to about 5-10% beyond the unstretched dimension in the circuit direction. When the circuit comprises a continuously knit stretch nylon yarn, stretch is limited to about 10-20% beyond the unstretched dimension in the circuit direction. When the circuit comprises a continuously knit 70 denier spandex yarn, single or double covered with a conductive nylon yarn, stretch is limited to about 50-100% beyond the unstretched dimension in the circuit direction.

In certain embodiments, the location to which the electrical signal is transmitted comprises an external device separate from the wearable device. For example, the external device can comprise an electronic display unit configured to display the transmitted value of a variable.

In certain embodiments, the circuit can be configured to conduct electrical signals in both directions along the circuit. In particular embodiments, the circuit can be configured to transmit power from a power source to a location on the wearable device.

In some embodiments, the wearable device comprises a compressive pressure device, and the variable comprises compressive pressure applied by the device. In some embodiments, the wearable device comprises a compressive pressure device, a sensor is configured to sense compressive pressure in an area of a body to which the device is applied, and the transmission circuit is configured to transmit an electrical signal representing an amount of compressive pressure sensed in the area of a body to an external electronic display unit. In particular embodiments, the compressive pressure device comprises an inner compressive pressure sleeve and an overlying outer compressive pressure wrap. In such an embodiment, the sensor can be located either (a) between the body and the sleeve, (b) within the sleeve (c) between the sleeve and the wrap, or (d) within the wrap. In either of these locations, the sensor is configured to sense an actual cumulative amount of compressive pressure applied by the sleeve and the wrap.

Some embodiments of the present invention include a body monitoring system comprising: a wearable device comprising an elastic fabric; and a circuit for conducting electrical signals comprising an electrically conductive silver yarn or a yarn coated with silver knitted into the device fabric in a vertical, horizontal, or angled direction. In such embodiments, the circuit can further comprise (a) a sensor circuit configured to sense a variable in an area of a body to which the device is applied, and (b) a transmission circuit configured to transmit an electrical signal representing a value of the variable in the area of the body to an external electronic display unit configured to display the transmitted value of the variable. In some such embodiments, the device fabric has an unstretched dimension in a direction of the circuit, and stretch beyond the unstretched dimension in the circuit direction is limited to provide sufficient circuit continuity for reliable conduction of the electrical signals. In some such embodiments, the wearable device comprises a compressive pressure device, the variable comprises compressive pressure applied by the device, and the transmission circuit is configured to transmit an electrical signal representing an amount of compressive pressure sensed in the area of a body to an external electronic display unit.

Some embodiments of the present invention include a body monitoring system comprising: a wearable device; a sensor configured to sense a variable in an area of a body to which the device is applied; and a transmission circuit comprising an electrically conductive yarn knitted into the device and configured to transmit an electrical signal representing a value of the variable in the area of a body to another location.

In some such embodiments, the sensor can further comprise a knitted cuff sensor. In one embodiment, the knitted cuff sensor comprises a three-layer capacitance type sensor comprising (a) an inner layer electrically conductive yarn, (b) a middle layer semi-conductive dielectric yarn, and (c) an outer layer electrically conductive yarn. In other such embodiments, the knitted cuff sensor comprises a two-layer capacitance type sensor comprising (a) an inner cuff layer and an outer cuff layer each comprising an electrically conductive yarn and (b) an electrically regulating dielectric material inserted between the inner and outer cuff layers. In yet other such embodiments, the knitted cuff sensor comprises a piezoelectric type sensor. In still other such embodiments, the knitted cuff sensor comprises a piezoresistive sensor comprising (a) an inner cuff layer and an outer cuff layer each comprising an electrically conductive silver yarn and (b) a piezoresistive semi-conductive polymer disposed between the inner and outer cuff layers.

In some embodiments, the body monitoring system can further include a cuff integrally knit into the wearable device, in which the cuff is configured to house a sensor. The sensor can comprise an electro-mechanical sensor, a capacitance sensor, or a piezoelectric sensor. In some embodiments, the body monitoring system can further include a pocket integrally knit into the wearable device, in which the pocket is configured to house the sensor. The sensor can comprise an electro-mechanical sensor, a capacitance sensor, or a piezoelectric sensor.

In some embodiments, the sensor can be securable to a hook-and-loop type fastener engagable with the wearable device. In such an embodiment, the sensor can comprise an electro-mechanical sensor, a capacitance sensor, or a piezoelectric sensor.

In some embodiments, the sensor can further comprise a sensor circuit printed onto a material comprising a hook-and-loop type fastener engagable with the wearable device. An electrically conductive yarn can be sewn through the material so that the yarn is conductively contactable between the printed sensor circuit and the transmission circuit in the wearable fabric.

In some embodiments, the wearable device comprises a compressive pressure device, the variable comprises compressive pressure, and the system can further comprise a pressurized cuff (a) having opposing ends releasably securable to each other, (b) adjustably positionable about the wearable device, and (c) having the sensor integrated into the cuff. When the pressurized cuff is adjusted about the wearable device to have the same initial compressive pressure as the wearable device, the sensor senses changes in actual applied pressure at an interface of the body area, the wearable device, and the pressurized cuff.

Features of a body monitoring system and/or method of the present invention may be accomplished singularly, or in combination, in one or more of the embodiments of the present invention. As will be realized by those of skill in the art, many different embodiments of a fabric, garment, and/or method according to the present invention are possible. Additional uses, advantages, and features of the invention are set forth in the illustrative embodiments discussed in the detailed description herein and will become more apparent to those skilled in the art upon examination of the following.

### BRIEF DESCRIPTION OF THE DRAWINGS

FIG. 1 is a view of a body monitoring system on a lower limb of a wearer in an embodiment of the present invention.

FIG. 2 is a view of a body monitoring system having knitted-in sensing and transmission circuits in an embodiment of the present invention.

FIG. 3 is a view of a body monitoring system having a knitted-in cuff and transmission circuit in an embodiment of the present invention.

FIG. 4 is a diagrammatic view of an electrically conductive yarn knitted as an angled transmission circuit in an embodiment of the present invention.

FIG. 5 is a diagrammatic view of an electrically conductive yarn laid in a knitted fabric structure as a transmission circuit in an embodiment of the present invention.

FIG. 6 is a view of a body monitoring system having a knitted-in pocket in an embodiment of the present invention.

FIG. 7 is a view of a compressive pressure device having a knitted-in cuff and transmission circuit in an embodiment of a body monitoring system of the present invention.

FIG. 8 is a view of a piece of material having a printed sensor circuit, engaged with a wearable fabric with a hook-and-loop type fastener, and conductively connected to a transmission circuit in the fabric in an embodiment of the present invention.

FIG. 9 is a view of an adjustable pressurized sensor cuff and a transmission circuit connecting the sensor cuff to a display unit in an embodiment of the present invention.

FIG. 10 is a view of an inner compression sleeve having integrally knit sensing and transmission circuits and an overlying compression wrap in an embodiment of the present invention.

### DETAILED DESCRIPTION

For the purposes of this description, unless otherwise indicated, all numbers expressing quantities, conditions, and so forth used in the description are to be understood as being modified in all instances by the term “about.” Accordingly, unless indicated to the contrary, the numerical parameters set forth in the following description are approximations that can vary depending upon the desired properties sought to be obtained by the embodiments described herein. At the very least, and not as an attempt to limit the application of the doctrine of equivalents to the scope of the invention, each numerical parameter should at least be construed in light of the number of reported significant digits and by applying ordinary rounding techniques.

Notwithstanding that the numerical ranges and parameters setting forth the broad scope of the described embodiments are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a stated range of “1 to 10” should be considered to include any and all subranges between (and inclusive of) the minimum value of 1 and the maximum value of 10.

As used in this description, the singular forms “a,” “an,” and “the” include plural referents unless the context clearly dictates otherwise. Thus, for example, the term “a yarn” is intended to mean a single yarn or more than one yarn. For the purposes of this specification, terms such as “forward,” “rearward,” “front,” “back,” “right,” “left,” “upwardly,” “downwardly,” and the like are words of convenience and are not to be construed as limiting terms. Additionally, any reference referred to as being “incorporated herein” is to be understood as being incorporated in its entirety.

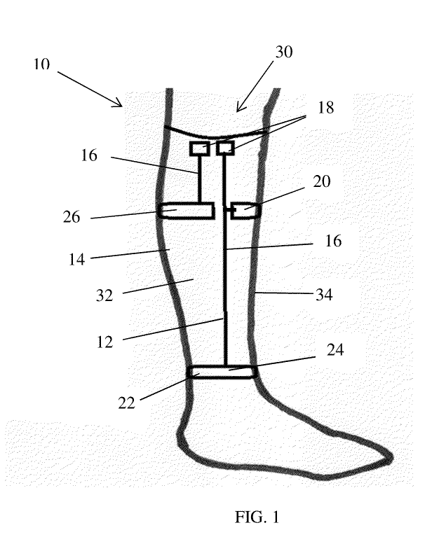

The present invention includes embodiments of a body monitoring system and/or method. FIGS. 1-10 illustrate such embodiments. In some embodiments, the body monitoring system 10 comprises a sensor configured to detect changes in one or more variables in a body. Various embodiments of the sensor can comprise electrical, mechanical, chemical, ultrasonic, acoustic, tactile, and/or other sensing mechanisms to monitor the intended variable(s). Embodiments of the body monitoring system 10 and/or method can be adapted to monitor variables in animate and/or inanimate bodies. Such variables include, for example, heartbeat, blood flow, pulse rate and quality, oxygenation, temperature, edema, body movements, and other physiological variables.

As shown in FIG. 1, the body monitoring system 10 can comprise an electrically conductive yarn 12 knitted into a fabric or garment 14 as a transmission circuit 16. The transmission circuit 16 provides a pathway for transmitting electrical signals representing a value of a monitored variable from a sensor located on the fabric/garment 14 to a display unit 18 where the variable value can be displayed. The sensor can comprise various forms and functionalities. For example, as illustrated in FIG. 1, the sensor can comprise the electrically conductive yarn 12 knitted into the fabric or garment 14 as a sensing circuit 20. In another embodiment, the sensor can be integrated into a cuff 22 that is knitted about the circumference of the tubular garment 14 (cuff sensor 24). In another embodiment, the sensor can be integrated into a pocket 26 that is knitted in a discrete area of the garment 14. The transmission circuit 16, sensing circuit 20, cuff sensor 24, pocket 26 adapted to contain a sensor, and display unit 18 are described in detail below. Other embodiments of the sensor and other aspects of the present invention are also described below.

In one illustrative embodiment, the body monitoring system 10 and/or method can comprise a system and/or method for monitoring compression in a body. Reference is made throughout this description to a body compression monitoring system 30 and/or method for purposes of illustration only. The inventive features of the present invention apply to systems and/or methods for monitoring a variety of variables other than compression and in different kinds of bodies.

As shown in FIG. 1, one embodiment of such a body compression monitoring system 30 can comprise a compressive pressure garment, wrap, bandage, or device 32 (collectively “compressive pressure device” or “device”) that incorporates into the system 30 an ability to monitor compressive pressure applied by the device 32 on a body. For purposes of illustration, the compressive pressure device 32 in FIG. 1 is configured to be worn on a person's lower limb 34. The body compression monitoring system 30 and/or method provides a mechanism for easily and accurately determining an actual amount of compressive pressure applied to an anatomical area by the compressive pressure device 32. The actual applied compressive pressure can be measured in mm Hg, for example. The body compression monitoring system 32 and/or method can further comprise the display unit 18, or mechanism for displaying measurements of the applied compressive pressure.

Various types of sensors configured to measure applied compressive pressure can be utilized in the body compression monitoring system 30 and/or method. A particular embodiment of such a body compression monitoring system 30 can include a single type of sensor or a combination of different types of sensors.

In some embodiments, the body monitoring system 10 can comprise a pathway from the sensor to the electronic display unit 18 where the value of a measured variable can be displayed. The pathway can have various dimensions and take various paths from the sensor to the display unit 18. The pathway can comprise a vertical path along the longitudinal axis of a wearable device 40, for example, along a wale 36 or a selected number of adjacent wales 36 in the knitted compressive pressure device/garment 32. For example, the pathway can extend from a sensor in the compressive pressure device 32, such as about an ankle, vertically to the display unit 18 at the top of the device 32. Measurements of applied compressive pressure by the sensor can be transmitted to the display unit 18 in the form of an electrical signal. Accordingly, the pathway can be referred to as a transmission circuit 16. Examples of vertical pathway transmission circuits 16 are shown in FIGS. 1, 2, 3, and 9.

The transmission circuit 16 can comprise electrically conductive yarn(s) 12. For example, the transmission circuit yarn 12 can be an electrically conductive silver yarn or a yarn coated with silver. Various commercially available silver yarns are useful in embodiments of the present invention. One preferred silver yarn is X-STATIC®, commercially available from Noble Biomaterials, Inc. (300 Palm Street, Scranton, Pa. 18505). The X-STATIC® silver yarn comprises 99.9% pure elemental silver and is highly electrically conductive, lightweight, flexible, stretchable, washable, and durable. In addition, the X-STATIC® silver yarn is a broad spectrum antimicrobial and odor eliminator useful in the care of wounds such as dermal ulcers.

The transmission circuit pathway 16 can be integrally knit into the wearable device 40 while the device 40 is being knit. During the process of knitting a tubular wearable device 40 on a circular knitting machine, yarns being knit for the device 40 are cut at a predetermined location about the device circumference. An electrically conductive yarn 12 is then picked up and dropped in for a selected number of cycles, for example, four cycles. After being knit for the selected number of cycles, the electrically conductive yarn 12 is dropped, and the yarn for knitting the device 40 is picked back up to continue knitting around the device circumference. These steps are repeated so as to construct the vertical transmission circuit 16, or stripe.

In some embodiments, the transmission pathway circuit 16 comprising the knitted electrically conductive yarn 12 can be knit on a flat bed knitting machine.

In another embodiment, the wearable device 40 can comprise polyester yarn, and the transmission pathway (circuit) 16 can comprise nylon yarn. Once the wearable polyester device 40 having a nylon yarn transmission pathway 16 is fabricated, the entire device fabric can be coated with silver or a silver composition. Because silver adheres to nylon but not to polyester, only the transmission pathway 16 is coated with the silver or silver composition. As a result, the nylon pathway is provided with an electrically conductive material to create the transmission circuit 16. To further assure that the silver-coated nylon stitches in the transmission circuit 16 are sufficiently packed together to provide a continuous circuit, the wearable device 40 can be heated. Heating the device 40 a particular amount shrinks the nylon yarn so as to further pack the nylon-silver yarns along the transmission circuit 16 for enhanced conductivity.

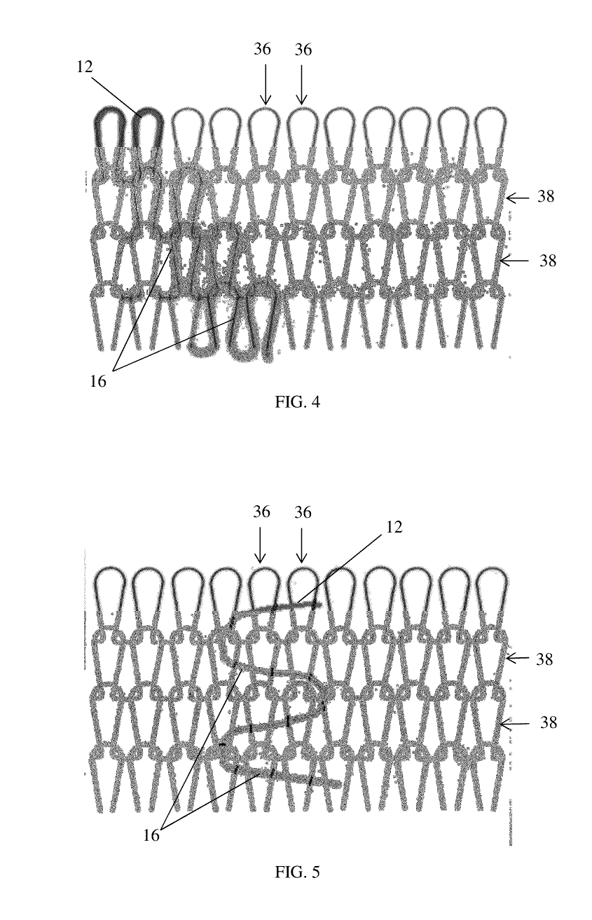

In embodiments of the body monitoring system 10 and/or method, transmission circuits 16 comprising electrically conductive yarns 12 can be knit in fabrics in any direction. That is, electrically conductive yarn circuits 12 can be knit vertically, horizontally, or at angles in a fabric. The direction and specific path of the transmission circuit 16 can be determined by the selection of stitch pattern and conductive yarn. An angled transmission pathway circuit 16 can be knit utilizing either cut yarns or a continuous yarn. To achieve an angled transmission circuit 16 utilizing cut yarns, the electrically conductive yarn 12 can be knit in a wale 36 offset from a previous wale 36 in successive courses 38 as the fabric is knitted in the vertical direction. FIG. 4 shows an example of an angled transmission pathway circuit 16 having a cut electrically conductive yarn 12. Such angled circuits 16 facilitate the use of sensors in various locations on a body, for example, about anatomical curvatures.

A horizontal transmission circuit 16 can be achieved by knitting the electrically conductive yarn 12 horizontally, or laterally, in a fabric along one or more courses 38. Alternatively, a continuous electrically conductive yarn 12 can be “laid in” a knitted fabric structure, for example, along one or more courses 38, to provide a horizontal transmission circuit 16. In certain embodiments, a continuous electrically conductive yarn 12 can be “laid in” a fabric structure so as to have changing directions to provide a transmission circuit 16 along a particular desired pathway. For example, FIG. 5 shows the electrically conductive yarn 12 “laid in” a fabric structure in a serpentine manner to provide the transmission circuit 16 at particular locations in the fabric. Providing the transmission circuit 16 at particular locations in this manner allows placement of sensors at desired locations in the fabric. The continuous electrically conductive yarn 12 can also be “laid in” a knitted fabric structure to provide an angled transmission pathway circuit 16.

In one aspect of the present invention, the electrically conductive transmission pathway, or circuit, 16 can be knit into a stretch fabric, that is, fabric having elasticity. Reliability of signal transmission along the pathway 16 depends, at least in part, on the continuity of the circuit 16. Circuit continuity relates primarily to yarn contact along the pathway 16. In some embodiments, circuit continuity can be enhanced by increasing yarn contact with a knit construction that packs stitch loops compactly together and/or shrinking a nylon-based pathway yarn by heating. In embodiments of an elastic fabric comprising the electrically conductive transmission pathway 16, circuit continuity can be further enhanced by limiting stretch in the direction of the pathway 16. In this way, reliable contact for conductivity can be maintained between stitches of the electrically conductive yarn 12 along the pathway 16.

For example, in embodiments of such an elastic fabric having the transmission circuit pathway 16 knit in the vertical direction, vertical stretch in the fabric can be limited. The limit of vertical stretch desirable in a stretch fabric depends on whether the electrically conductive yarn 12 in the transmission pathway 16 is knit in a cut manner or in a continuous, uncut manner.

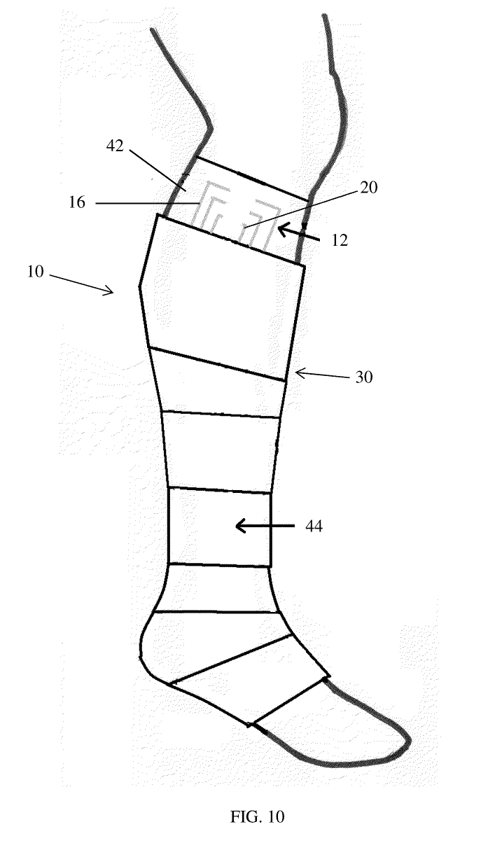

In embodiments in which the electrically conductive yarn 12 is knit in a cut manner, stretch in the direction of the transmission pathway 16 is preferably limited to about 5-10% beyond the unstretched, or resting, dimension of the fabric in the pathway direction. For example, in a rectangular, or elongated, compressive pressure wrap 44 (as shown in FIG. 10) having the transmission pathway 16 knit in the vertical direction along the length of the wrap 44, vertical (or longitudinal) stretch is preferably limited to about 5-10% beyond the unstretched length of the wrap 44. In “cut yarn” knitting on a circular knitting machine, the electrically conductive yarn 12 is brought up in one or more needles to the tuck height where the yarn 12 is cut. The cut electrically conductive yarns 12 in adjacent wales 36 are tightly knit, or packed together, so as to provide continuous contact between the cut yarns 12 to form the transmission circuit 16 in the vertical direction. It was further discovered that washing a fabric having a cut yarn transmission pathway 16 causes the tails of the cut yarns 12 to draw inward toward adjacent cut yarns 12 to improve electrical conductivity along the pathway 16.

In embodiments in which the electrically conductive yarn 12 is knit in a continuous, uncut manner, the amount of stretch in the direction of the transmission pathway 16 permissible to maintain sufficient electrical conductivity depends on the type of conductive yarn 12. For example, when the electrically conductive yarn 12 is a conductive stretch nylon, stretch in the direction of the transmission pathway 16 is preferably limited to about 10-20% beyond the unstretched, or resting, dimension of the fabric in the pathway direction. Additional permissible stretch can be achieved by utilizing yarn having a higher stretch modulus. For example, when the electrically conductive yarn 12 is a 70 denier spandex yarn, single or double covered with a conductive nylon yarn, stretch in the direction of the transmission pathway 16 can be about 50-100% beyond the unstretched dimension of the fabric in the pathway direction without diminishing conductivity sufficient for signal transmission.

Although stretch in the direction of the knitted transmission pathway 16 is preferably limited, embodiments of such elastic fabrics can have substantial stretch in the direction opposite the direction of the transmission pathway 16 without affecting transmission of an electrical current signal along the pathway 16. As discussed, preferred limitations of stretch depend on the direction of the transmission pathway 16 and the construction of the pathway circuit 16. For example, in an elastic fabric having a pathway 16 knit in the vertical direction, the fabric can be stretched in the horizontal direction without affecting transmission of an electrical current signal along the vertical pathway 16.

The vertical pathway transmission circuit 16 can be knit using various knit patterns. In a preferred embodiment, the vertical pathway transmission circuit 16 is knit in a rib pattern. In a rib stitch pattern, wales 36 are alternated between the face of the fabric and the back of the fabric. The rib pattern can be two, threes, or four needles (or wales 36) wide, for example. In the transmission circuit 16 knit in a rib pattern, silver can be plated on one side of the rib, preferably the back side of the rib. The rib pattern can be either an elastic or a nonelastic rib pattern, which can be programmed into the knitting machine.

Conductivity properties in the knitted transmission circuit 16 and in the knitted sensing circuit 20 can vary depending on a number of factors, including the type of electrically conductive yarn 12, yarn size (denier), yarn construction, amount of yarn in a given area (yarn/fabric density), and stitch pattern. That is, such factors can be balanced in a fabric structure to achieve conductivity in the circuit 16, 20 suitable for reliably transmitting signals. For example, an electrically conductive silver yarn has different conductivity properties than an electrically conductive stainless steel yarn. A knitted-in circuit 16, 20 comprising a yarn having a first denier has different conductivity properties than a knitted-in circuit comprising a yarn having a second, different denier. Yarn sizes suitable for reliable signal transmission conductivity in some sensor applications include yarns in the range of about 70 denier to about 370 denier. Reliable signal transmission conductivity may also be achieved in more sheer fabrics having smaller denier yarns. As an example, a single 70 denier silver yarn provides for transmission of a reliable electrical signal in some sensor applications/embodiments. In other applications/embodiments, two 70 denier silver yarns twisted together to form a 140 total denier yarn provides for transmission of a reliable electrical signal. In still other embodiments, the electrically conductive yarn 12 can be a covered stretch yarn.

A larger amount, or density, of yarn 12 in a knitted-in circuit generally exhibits greater conductivity than a smaller density of yarn 12. A knitted-in circuit 16, 20 comprising a standard single jersey stitch pattern has different conductivity properties than a knitted-in circuit 16, 20 comprising a different stitch pattern. Likewise, different selections of a rib pattern may affect conductivity in the knitted-in circuit 16, 20. For example, a 2×2 rib selection may have different conductivity than a 1×1 rib selection. Thus, by altering the yarn type, size, amount, and pattern in the knitted circuit 16, 20, the flow of electrical signals can be controlled. As a result, the type of variables being monitored and the manner in which those variables are monitored can be controlled.

In addition, various combinations of such conductivity factors can be utilized in different sections of the garment 14. In this way, the flow of electrical signals/current can be controlled as desired for monitoring multiple variables in the same garment 14. Similarly, the dimensions of the knitted-in circuit 16, 20 can be varied by programming the knitting machine to knit different widths, lengths, and/or shapes of the circuit 16, 20. Circuits 16, 20 having different dimensions in the fabric/garment 14 can have different conductivity properties that can be utilized for different purposes in the same fabric/garment 14.

During the process of knitting the body monitoring system 10, such as in the process of knitting the compressive pressure device 32, the electrically conductive yarns 12 knit in the vertical transmission circuit 16 are preferably “packed” together vertically. That is, the electrically conductive yarns 12 are knit tightly so that the stitch loops in adjacent courses 38 along a particular wale 36 are compacted together. In this way, the electrically conductive yarns 12 in adjacent courses 38 have sufficient contact to provide a continuous circuit. Such a continuous circuit allows transmission of an electrical signal representing a compressive pressure measurement from a sensor to another location, such as the electronic display unit 18.

In some conventional tubular compressive pressure garments, yarn stitches in the upper portion of the garment are knit more loosely than in the rest of the garment to provide a more tailored fit about a larger upper part of the limb on which it is to be worn. However, in the compressive pressure device 32 having the vertical transmission circuit 16, yarns 12 in the circuit 16 are preferably knit tightly in the entire extent of the circuit 16 to provide sufficient yarn contact throughout the circuit 16 for reliable signal transmission.

The transmission circuit 16 is connected to the sensor with an interface appropriate for the type of sensor. For example, a different type of interface can be utilized to connect the transmission circuit 16 for each of the knitted cuff sensor 24, a stand-alone electrically conductive yarn sensor, a separate electro-mechanical, capacitance, or piezoelectric sensor housed within the cuff 22 or pocket 26, or other sensor. In each instance, the transmission circuit connection with the sensor is configured to allow transmission of an electrical signal representative of a value of a sensed variable to the display unit 18 where the value of a sensed variable can be displayed.

The number of transmission circuits 16 in the wearable device 40 can vary, depending on the number of sensors in the device 40 from which measurements of a variable are to be transmitted. Transmission circuits 16 can be placed at different locations about the wearable device 40 as desired. For example, three vertical transmission pathways 16 can be placed on two different sides of the tubular device 40, one circuit 16 each for a sensor on the lateral aspect and the medial aspect of the instep, ankle, and calf.

While the knitted-in transmission circuit 16 is a preferred mechanism for transmitting a measure, or value, of a variable, such as an amount of applied compressive pressure, to the display unit 18, other mechanisms are contemplated. For example, an electrically conductive wire, such as a copper wire, can be utilized to transmit signals representing measurements of the variable from the sensor to the display unit 18. In such an embodiment, the copper wire can be integrated into the fabric of the wearable device 40, either by knitting the wire in the fabric 40 or by laying in the wire during construction of the device 40. Alternatively, such a wire can be attached externally to the wearable device 40.

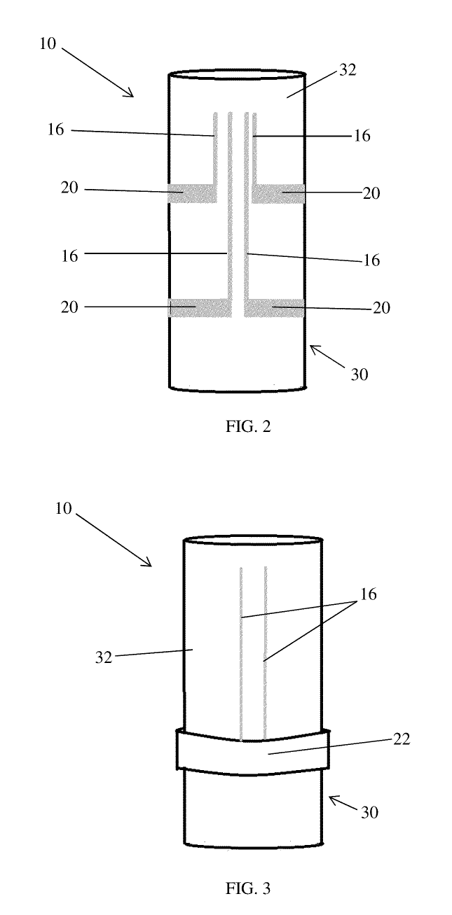

In some embodiments of the body monitoring system 10 and/or method, the sensor can be a knitted-in sensor circuit 20. The knitted-in sensor circuit 20 can be constructed using electrically conductive yarn 12 in a manner similar to the knitted-in transmission circuit 16. An advantage of the knitted-in sensor circuit 20 is that it can be knit to have various shapes and/or dimensions and placed in desired locations throughout the wearable device 40. Configuration and positioning of the knitted-in sensor circuit 20 can readily be accomplished by programming a knitting machine. One preferred shape of the knitted-in sensor circuit 40 is a rectangle, positioned horizontally about a tubular wearable device 40, such as the compressive pressure garment 32, as shown in FIGS. 1 and 2. The knitted-in sensor circuit 20 can be adapted to measure one or more variables, such as applied compressive pressure, at various points throughout the sensor dimension. Such a sensor having a horizontal orientation about a wearer's limb can thus provide measurements of the variable(s), such as applied compressive pressure, about an entire anatomical plane.

The sensor circuit 20 can be knit into the fabric of the wearable device 40. In one embodiment, the wearable device can comprise a compression sleeve 42, as shown in FIG. 10. In this way, when the sleeve 42 is worn without an overlying application, such as a compression wrap 44, the compressive pressure applied by the sleeve 42 can be measured. In addition, when the wrap 44 or other compressive pressure device is applied on top of the sleeve 42, the cumulative compressive pressure of the inner sleeve 42 and the outer wrap 44 or device can be measured.

In some embodiments, the knitted-in circuit can be a circuit that only transmits an electrical signal. In other embodiments, the knitted-in circuit can be a circuit that only senses a variable in the area of a body to which the wearable device 40 is applied. In yet other embodiments, the knitted-in circuit can be both the sensing circuit 20 and the transmission circuit 16.

In another aspect of the present invention, certain knitted-in circuits 16, 20 may be configured to transmit power from a power source to a device within or on a fabric, garment, or bandage. Power transmitted from an external power source to a location in the fabric/garment 14 can be utilized for various purposes. Such purposes can include, for example, direct electrical stimulation therapy, heating the fabric, or powering a device, such as a transcutaneous electrical nerve stimulator unit or a miniature air pump.

In some embodiments, the wearable device 40 can comprise the electrically conductive transmission pathway 16 constructed so as to allow electrical transmission in both directions along the pathway 16. In such embodiments in which an electrical current can travel in both directions, one part of the circuit 16 can be configured to transmit an electrical signal representing the value of a sensed variable from a sensing area on the body to the external electronic display unit 18, and another part of the circuit 16 can be configured to transmit an electrical current, such as powerable current, from a first location in the wearable device 40 to second location in the device 40 or from a location separate from the device 40 to a desired location in the device 40.

One sensor comprises the cuff 22 integrally knit into the fabric of the wearable garment or device 40, such as the compressive pressure device 32 shown in FIGS. 1 and 3. The cuff sensor 24 comprises electrically conductive yarns 12 capable of sensing a variable, such as the amount of compressive pressure being applied. In some embodiments, the knitted cuff sensor 24 is constructed to have three knitted fabric layers—a first layer comprising a base fabric layer of the wearable device 40; a second layer comprising an inside layer of the cuff 22; and a third layer comprising an outside layer of the cuff 22. That is, the cuff 22 can be constructed to overlie the first, device layer. The second, inside layer of the cuff 22 lies adjacent the first, device layer. The cuff 22 can have a length such that it can be folded over onto itself, such that the third, outside layer of the cuff 22 is adjacent the second, inside cuff layer.

In one embodiment, the knitted cuff sensor 24 comprises a capacitance type sensor. In one knitted cuff, capacitance type sensor, the first, base layer of the wearable device 40 comprises an inner electrically conductive yarn 12. The second, inside layer of the cuff 22 comprises a semi-conductive yarn. And, the third, outside layer of the cuff 22 comprises an outer electrically conductive yarn 12. With an electric current running through the inner and outer conductive yarns 12, the separation between the first and third fabric layers can be measured to provide a capacitance value for the measurement area. Such a capacitance value can be correlated to, for example, an amount of compressive pressure being applied by the wearable device 40. A change in capacitance value can thus be correlated with an amount of change in applied compressive pressure.

The electrically conductive yarn(s) 12 in both the first, base layer of the device 40 and in the third, outer cuff layer can comprise yarn such as silver yarn or stainless steel yarn. One preferred silver yarn for the knitted cuff sensor is X-STATIC®, commercially available from Noble Biomaterials, Inc. Alternatively, the first, base layer of the device 40 and the third, outer cuff layer can comprise nylon and polyester yarns. The layers can be constructed so that the nylon yarns are in a particular pattern configured for sensing an area of compressive pressure. A conductive silver composition can be applied to the first and third layers, whereby the silver composition adheres to the nylon but not to the polyester. In this way, the silver-coated nylon yarns can function to carry an electrical current and act as capacitance-based compression-sensing bars, or nodes.

The knitted cuff sensor 24 can be constructed so that the range, or spread, of electrical conductivity (sensitivity) in a sensing area is broad enough to reliably detect differences in a variable, such as compression, represented by an electrical signal. For example, in some embodiments, the range of electrical sensitivity can be between about 5-15 kOhms. In other embodiments, electrical conductivity/sensitivity can comprise other ranges, depending on the variable being sensed. In testing, it was discovered that some silver yarns are too conductive to transmit electrical signals in such a desired sensing range. The preferred X-STATIC® silver yarn provides a range of electrical conductivity/sensitivity that allows sensing variables in embodiments of the present invention.

In another embodiment of a knitted cuff, capacitance type sensor, each of the inside layer and the outside layer of the cuff 22 comprises the electrically conductive yarn 12. An electrically regulating dielectric insulator material can be inserted between the two layers of the cuff 22. In this configuration, capacitance between the two electrically conductive layers of the cuff 22 can be measured as a function of compressive pressure applied by the compressive pressure device 32. That is, as the limb 34 on which the compressive pressure garment or device 32 is being worn swells or otherwise changes shape, increasing pressure at the interface between the limb 34 and the garment/device 32 will likewise be applied to the interface of the garment/device 32 with the knitted cuff 22. In this way, the cuff sensor 24 can sense changing pressure applied to the underlying limb 34.

In another embodiment, the knitted cuff sensor 24 comprises a piezoelectric type sensor. A piezoelectric pressure sensor measures changes in pressure by converting those changes to an electrical charge. In one knitted cuff, piezoelectric type sensor, the first, base layer of the wearable device 40 comprises a non-conductive plate portion integrated with or attached to the layer. The second, inside layer of the cuff 22 comprises a conductive material, for example, a copper wire knit into the fabric of the second layer. And, the third, outside layer of the cuff 22 comprises a non-conductive plate portion integrated with or attached to that layer. The non-conductive plate portions can be a plastic material, for example. As the two non-conductive plate portions move in relation to each in response to changing compressive pressure exerted by the device 40, the force field between the plates changes. The change in pressure between the plates can be measured as a change in electrical charge carried along the copper wire.

In another embodiment, the knitted cuff sensor 24 comprises a piezoresistive type sensor. In such a sensor 24, the first, base layer of the wearable device 40 comprises an inner electrically conductive yarn 12. The second, inside layer of the cuff 22 comprises a piezoresistive semi-conductive polymer. The piezoresistive material comprises an electrical resistivity that varies inversely with pressure exerted on the material. And, the third, outside layer of the cuff 22 comprises an outer electrically conductive yarn 12. The inner and outer electrically conductive yarn(s) 12 in the first and third layers can comprise any electrically conductive yarn, and preferably is a silver yarn. In such a piezoresistive sensor, a change in compressive pressure applied by the device 40 causes a change in resistance between the two layers (first and third layers) comprising electrically conductive yarns 12. The change in resistance can be converted to an electrical signal representative of a correlated amount of applied compressive pressure.

Embodiments of the body monitoring system 10 can have one or more cuff sensors 24, as shown in FIGS. 1 and 4, knit into the wearable device 40. The cuff(s) 22 can be knit at location(s) along, for example, the compressive pressure garment/device 32 desired for measuring applied compressive pressure at such location(s). For example, cuffs 22 can be knit at the calf, ankle, and/or instep in the compressive pressure device 32 designed for the lower limb 34. Embodiments of the body monitoring system 10 having the knitted cuff sensor 24 can be manufactured all in one step, for example, on a circular knitting machine. That is, the circumferential cuff 22 can be integrally knit while the wearable device is being knit.

In a one embodiment, the compressive pressure device 32 and cuff 22 can be knit with a Lonati Model GL615 electropneumatic single cylinder circular knitting machine. This machine has a 168-needle cylinder containing 3¾ inch medium butt and short butt needles typically used for knitting socks. The machine includes a single main feed with eight yarn finger selections, one elastic selection at the main feed, and five pattern feeds. One elastic station has two elastic selections.

During knitting of the compressive pressure garment 32, the cuff 22 can be knit at a desired location. Beginning with a circular knitting motion, the cuff 22 can be knit by loading the needles using a 1×1 selection at the main feed for one revolution of the needle cylinder, with a yarn delivered by one of the yarn fingers at the main feed. In the second revolution, all needles come up to knit height for one revolution to lock the stitches onto the needles. In the third revolution, the cylinder needles change to a 1×1 selection opposite from selection in the first revolution, and the dial jacks are loaded with yarn by moving out between the cylinder needles that are down for one revolution.

The knitting machine can be programmed to operate as in the third revolution for a set number of courses 38 to obtain a desired length for the cuff 22. After a set number of courses 38 for the cuff 22 has been knit, dial cams for controlling the dial jacks are activated. This causes the dial jacks to move our over the cylinder needles so that yarn being held by the dial jacks is transferred back onto the cylinder needles to complete the cuff 22. In this manner, the knitted cuff sensor 24 can be integrally knit into the compressive pressure device 32. Various yarns and stitch patterns can be knitted into the garment device 32 and cuff 22 sections to create various types of sensors as described herein. In certain embodiments, different yarns and stitch patterns can be used for each of the inside layer and the outside layer of the cuff 22.

In some embodiments of the body monitoring system 10 and/or method, the sensor can comprise the electrically conductive yarn 12 knit into the wearable device. For example, the compressive pressure device 32 can be knit such that the electrically conductive yarn(s) 12 are positioned at desired locations for measuring compressive pressure. An amount of applied compressive pressure can be sensed by the yarn(s) 12 and converted to an electrical signal representative of an amount of pressure. In one such embodiment, the electrically conductive sensor yarn(s) 12 can be knit into an inner surface of the fabric of the compressive pressure device 32 so that those yarns 12 are in contact with an underlying body. In another embodiment, the cuff 22 can comprise the electrically conductive yarn circuit 20 configured to sense one or more variables in a body. The sensor circuit 20 in the cuff 22 can be connected to the knitted transmission pathway circuit 16.

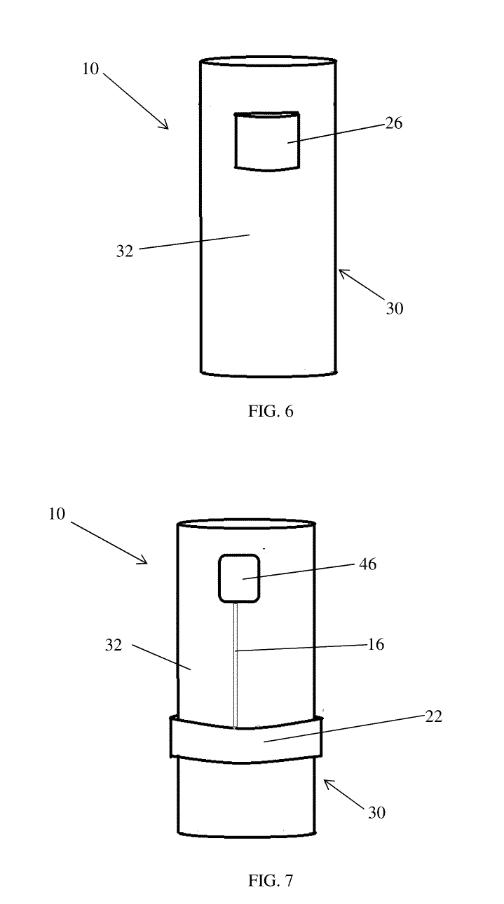

Embodiments of the body monitoring system 10 can have one or more pockets 26, as shown for example in FIGS. 1 and 6, knit into the wearable device 40. A separate sensor can be placed into, or housed in, the pocket 26. One advantage of the body monitoring system 10 in which a separate sensor is placed in the pocket 26 is that stretching of other layers of the wearable device 40 has minimal effect, or no effect, on the measurement of the variable(s) at the sensor location. The pocket(s) 26 can be knit at location(s) along a compressive pressure garment/device 32 desired for measuring applied compressive pressure at such location(s). For example, pockets 26 can be knit at the calf, ankle, and/or instep in the compressive pressure device 32 designed for the lower limb 34. Accordingly, actual compressive pressure at each of the locations at which a sensor is located can be accurately measured.

Embodiments of the body monitoring system 10 having the knitted pocket 26 can be manufactured all in one step, for example, on a circular knitting machine. That is, the pocket 26 can be integrally knit while the compressive pressure garment/device 32 is being knit. For example, using the Lonati circular knitting machine described herein, the pocket 26 can be knit at a desired location during knitting of the compressive pressure garment 32. To construct a knitted-in pocket 26, the needle cylinder moves from a circular motion into a reciprocated motion using medium butt needles. Needle lifters are used to raise the needles one at a time, one in each direction of reciprocation, and needle droppers are used to lower the raised needles down to knitting height out of action. The machine then reciprocates knitting on the medium butt needles only for a set number of courses to form the pocket 26. By holding the needle lifters and needle droppers out of action and open on each side, a seamless pocket 26 can be knitted. In this manner, the pocket 26 can be knitted either on the inside surface or on the outside surface of the compressive pressure device 32.

A compressive pressure sensor can be placed inside the pocket 26 for monitoring compressive pressure applied at the pocket location. In addition to sensors, various other devices such as, pumps, wireless transmitters, batteries, and/or other components related to a compressive pressure device 32 can be placed inside the pocket 26. One advantage of housing a device inside the pocket 26 is that the sensor or component is securely maintained in a desired position, while the sensor or component does not touch the skin of the wearer.

In similar fashion as the pocket 26, the cuff 22 integrally knit into the compressive pressure device 32 according to a method of the present invention can serve to house a separate compressive pressure sensor or other device. When the cuff 22 is utilized to hold a separate compressive pressure sensor in position in a desired location, the cuff 22 is preferably a non-sensing cuff. That is, in this application, the cuff 22 is knit without electrically conductive yarns 12.

In some embodiments of the body monitoring system 10 and/or method, the sensor can be an electro-mechanical sensor. The separate electro-mechanical sensor can be placed into, or housed in, the pocket 26 and/or the cuff 22 knit into the wearable device 40. Accordingly, a value of a variable at each of the locations at which the electro-mechanical sensor is located can be accurately measured.

One electro-mechanical sensor useful in a body monitoring system 10 and/or method is a flat force sensor. For example, the flat force sensor can be a force-sensing resistor (FSR) that exhibits a decrease in resistance when there is an increase in the force applied to the resistor. Thus, the resistor-sensor is able to detect force or pressure, including compressive pressure applied by the compressive pressure garment/device 32. In one embodiment, the resistor-sensor can comprise a polymer thick film (PTF) optimal for sensing an applied force ranging from a few dozen grams to over 10 kg. The resistor-sensor is preferably an elongated strip, approximately ½-¾ inch wide, and can have an active sensing area that is about ¼ inch wide. The resistor-sensor strip is desirably thin (for example, about 0.025 inch) and flexible, yet does not appreciably compress when pressure is applied. Such a force-sensing resistor is commercially available from Interlink Electronics, 546 Flynn Road, Camarillo, Calif., 93012 (www.interlinkelectronics.com). As a result, the force-sensing resistor sensor can be inserted flat or with only a slight curve within the cuff 22 or pocket 26 on the compressive pressure device 32 so as to maintain accuracy of pressure measurements.

In some embodiments of the body monitoring system 10 and/or method, the sensor can be a capacitance sensor. The separate capacitance sensor can be placed into, or housed in, the pocket 26 and/or the cuff 22 knit into the wearable device 40. Accordingly, a value of a variable at each of the locations at which the capacitance sensor is located can be accurately measured.

A capacitance sensor typically comprises two parallel plate conductors and an insulator between the two plates. Capacitance is directly proportional to the surface area of the parallel plates and inversely proportional to the separation distance between the plates or the displacement of one plate relative to the other plate. Capacitance can be calculated as the area of overlap of the two plates multiplied by a dielectric constant (relative static permittivity) and an electric constant, divided by the separation between the plates. Thus, a particular separation between two plates can be measured as a capacitance value for the measurement area. Such a capacitance value can be correlated to an amount of compressive pressure being applied by the compressive pressure device 32. A change in capacitance value can thus be correlated with an amount of change in applied compressive pressure.

In some embodiments of the body monitoring system 10 and/or method, the sensor can be a piezoelectric sensor. A piezoelectric pressure sensor measures changes in pressure by converting those displacement changes to an electrical charge. The separate piezoelectric sensor can be placed into, or housed in, the pocket 26 and/or the cuff 22 knit into the wearable device 40. Accordingly, a value of a variable at each of the locations at which the piezoelectric sensor is located can be accurately measured.

As described herein, the body monitoring system 10 and/or method can comprise the cuff 22 integrally knit with the compressive pressure device 32 in such a manner that the cuff 22 itself comprises the sensor. Alternatively, the cuff 22 and/or the pocket 26 can be knit into the wearable device 40 and configured to hold a separate sensor inside the pocket 26 or cuff 22. The separate sensor can be an electro-mechanical sensor, a capacitance sensor, or a piezoelectric sensor. Similarly, the non-sensing cuff 22 and/or pocket 26 can be adapted to house other devices and/or components related to a particular wearable device 40. For example, in one particular embodiment, the knitted-in cuff 22 can be constructed to hold an adjustable air bladder, as shown in FIG. 7. The air bladder housed in the knitted-in cuff 22 can be connected to an air pump 46 via the transmission circuit 16.

In some embodiments, the sensor can be attached to the wearable device 40 using a hook-and-loop type fastening system. For example, a surface of the wearable device 40 can comprise one portion 54 of a hook-and-loop type fastener that is engagable with a mating portion 56 of such a fastener. The sensor can be secured to a strip of material comprising the mating portion 56 of the fastener. By attaching the sensor-containing strip of the mating portion 56 to the hook-and-loop fastening enabled wearable device 40, the sensor can be reliably secured to the device 40.

The wearable device 40 using a hook-and-loop type fastening system can include an engagable portion 54 of the fastening system over the entire surface of the device. In this way, a mating portion 56 of the fastener having an attached sensor can be positioned for measuring the variable(s) at any location on the wearable device 40. Alternatively, the wearable device 40 can include an engagable portion 54 of the fastening system at selected locations on the device 40 at which variable measurements are desired. For example, an engagable portion 54 of the fastening system may be incorporated at the instep, ankle, and calf areas of the compressive pressure device 32 for measuring applied compressive pressure in those areas. In one particular variation, the entire surface, or selected areas, of the compressive pressure device fabric can be bulked by heat treatment to form a thin “blanket” of filaments. That “blanket” of filaments establishes a large number of loops which can be made to serve as the loop portion of a hook-and-loop type fastening system. Nylon yarns are particularly amenable to forming a blanket of loops when heated in this manner.

Depending on the type of sensor, the sensor may be attached using a hook-and-loop type fastening system to the inner surface (adjacent a wearer's skin) or to the outer surface of the wearable device 40. One advantage of attaching a sensor to the compressive pressure device 32 using a hook-and-loop type fastener is that the sensor-containing strip portion is pliable about the anatomical contours of a wearer's limb, such as about the ankle.

In one aspect of the present invention, changes in a variable are either sensed in the form of an electrical signal or are converted to an electrical signal. The electrical signal can be transmitted to the electronic display unit 18.

In some embodiments of the body monitoring system 10 and/or method, the sensor can comprise the electrical sensor circuit 20 adapted to measure one or more variables. The electrical sensor circuit 20 can be configured to amplify and filter a sensed variable signal to enhance and “clean up” the signal. The “cleaned up” signal can then be sampled by an analog-to-digital converter, and curve-fitting equations can be utilized to convert the digital signal into a measurement of the variable, for example, a measurement of force.

In some embodiments of the body monitoring system 10 and/or method, the electrical signal transmitting a variable measurement can be transmitted via the transmission circuit 16 adapted for such transmissions. In some embodiments, the sensing circuit 20 and/or the transmission circuit 16 can be printed or etched onto a portion of a piece of material 50 comprising a hook-and-loop type fastener engagable with the wearable fabric 40. Such printed circuits 20, 16 can then be secured to the wearable fabric 40 using the hook-and-loop type fastening system.

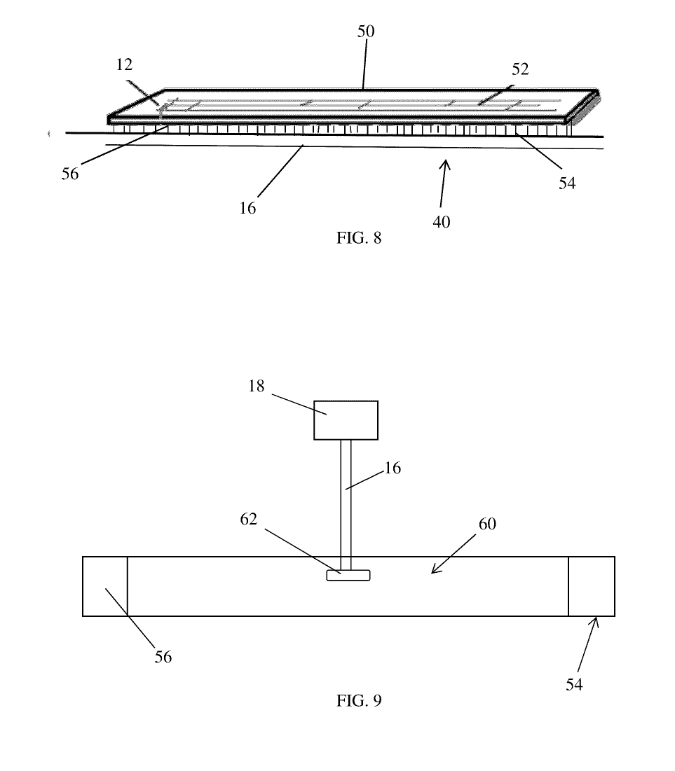

For example, as shown in the embodiment in FIG. 8, a piece of material 50 comprising the first portion 54 of a hook-and-loop type fastener can be printed with a sensor circuit 52 configured to sense a variable or parameter in/on a body. An electrically conductive yarn 12 can be sewn at a selected location in the sensor circuit 52 through the material 50 to expose the sewn conductive yarn 12 in the engagable first portion 54 of the hook-and-loop type fastener. The wearable fabric 40 can be constructed to comprise the second portion 56 of the hook-and-loop type fastener engagable with the first portion 54 of the fastener on the circuit material 50. The printed sensor circuit material 50 can be attached to the wearable fabric 40 at a location such that the exposed conductive yarn 12 on the circuit material 50 makes conductive contact with the transmission pathway circuit 16 in the fabric of the wearable device 40. In some embodiments, the body monitoring system 10 and/or method can comprise the body compression monitoring system 30 and/or method, the wearable device 40 can comprise the compressive pressure garment or device 32, and the printed sensor circuit 52 can be configured to sense applied compressive pressure.

The printed sensor circuit 52 can be placed against a body area to sense a variable. The sensor circuit material 50 and the printed sensor circuit 52 thereon can comprise a variety of shapes and/or dimensions. As a result, the printed sensor circuit 52 can be placed at various locations on a body while being connected to the transmission pathway circuit 16 in the wearable device 40. In this way, the printed sensor circuit 52 can be utilized to sense variables at particular locations in/on the body without having to vary the pathway of the transmission circuit 16. That is, one transmission pathway circuit 16 can be utilized to transmit signals from various, adjustable locations.

In some embodiments, the printed sensor circuit material 50 can be attached to a stretch fabric. Since the printed sensor circuit material 50 comprises a separate component from the knitted fabric to which it is attached, when the fabric is stretched, movement of the printed sensor circuit 52 is minimized and the ability of the printed circuit 52 to sense variables in a body is not affected. In some embodiments, the printed circuit 52 can be constructed so as to sense variable(s) and/or accept power from a power source.

In another aspect of the present invention, the body monitoring system 10 and/or method can comprise an adjustable pressurized cuff 60 that is wearable about a body area. As shown in FIG. 9, the pressurized cuff 60 can comprise an elongated piece of material, the ends of which can be overlapped onto each other and releasably connected. In the embodiment shown in FIG. 9, the cuff 60 can comprise a first portion 54 of a hook-and-loop type fastener on one end and a second, mating portion 56 of the hook-and-loop type fastener on the opposite end. The first and second hook-and-loop type fastener portions 54, 56, respectively, can be situated on the ends of the cuff 60 so that when the cuff 60 is wrapped about a circumferential surface, the ends of the cuff 60 can be releasably secured to each other about the surface so as to provide different lengths, and different amounts of tension, about the surface. The pressurized cuff 60 can further comprise one or more pressurized cuff sensors 62 integrated into the cuff 60 configured to sense pressure being applied by the cuff 60. The pressurized cuff sensor(s) 62 can be operably connected to the transmission circuit 16 that leads to the display unit 18.

In operation, the pressurized sensor cuff 60 can be placed about the person's limb 34, so as to overlie the compressive pressure garment 32 on the limb 34. The compressive pressure garment 32 can have a predetermined amount of compressive pressure when applied, for example, as calibrated on a tube having a particular circumference. Likewise, the pressurized sensor cuff 60 can be calibrated to provide a predetermined amount of compressive pressure when applied with a certain amount of tension. The pressurized sensor cuff 60 can be applied over the limb 34 and garment 32 so as to provide the same amount of compressive pressure as the amount rated for the garment 32. The amount of compressive pressure applied by the pressurized sensor cuff 60 can be adjusted by tightening or loosening the cuff 60 and securing the cuff 60 onto itself using the hook-and-loop type fastener system on the ends of the cuff 60. The amount of compressive pressure applied by a certain degree of tension on the cuff 60 can be monitored by reading the compressive pressure value displayed by the display unit 18. Thus, for a compressive pressure garment rated for 30 mm Hg pressure, for example, the pressurized sensor cuff 60 can be adjusted about the limb 34 and underlying garment 32 so that the display unit 18 displays an initial compressive pressure value of 30 mm Hg.

As the amount of applied compressive pressure on the limb 34 changes, the amount of pressure within the pressurized sensor cuff 60 changes proportionately. For example, as the girth of the limb 34 increases due to increasing edema, the amount of compressive pressure being applied by the pressure garment 32 and by the pressurized sensor cuff 60 increase. Accordingly, the display unit 18 will display an increasing compressive pressure value, thereby alerting the patient and/or caregiver that the actual applied compressive pressure may be too high for therapeutic purposes.

The sensor can be placed between a patient's body and the wearable device 40, such a compressive pressure sleeve, such as the sleeve 42 shown in FIG. 10. In such a configuration, the sensor can measure the cumulative, or total, compressive pressure applied by both the sleeve 42 and any overlying garment, such as the compression wrap 44. Alternatively, the sensor can be placed between the sleeve 42 and the overlying compression wrap 44 such that the sensor measures only the compressive pressure applied by the overlying wrap 44. In such an embodiment, the sleeve 42 having a predetermined applied compressive pressure, for example, about 5 mm Hg compressive pressure, can be placed on the patient's limb 34. The sensor can be attached to the outer surface of the sleeve 42 prior to the sleeve 42 being placed on the patient's limb 34, or the sensor can be placed on the outer surface of the sleeve 42 after the sleeve 42 is placed on the patient's limb 34. The wrap 44 can then be applied over the sensor and sleeve 42 such that the sensor is positioned between the inner sleeve 42 and the outer wrap 44. Once the outer wrap 44 is applied, the sensor can measure the compressive pressure applied by the outer wrap 44. By knowing the actual pressure applied by the wrap 44 on the patient's limb 34, the wrap 44 can be loosened or tightened to achieve a desired cumulative, or total, compressive pressure applied by both the inner sleeve 42 and the outer wrap 44. For example, if the total compressive pressure desired for treatment of a venous leg ulcer underneath the sleeve 42 and wrap 44 combination is 40 mm Hg pressure, the wrap 44 can be adjusted to provide 35 mm Hg pressure as measured by the sensor, which combined with the 5 mm Hg pressure provided by the sleeve 42 achieves the desired cumulative compressive pressure. In this way, the actual initial compressive pressure applied by the wrap 44, or sleeve 42 and wrap 44, for a particular treatment can be achieved with some certainty.

In another embodiment in which the sensor is place between the sleeve 42 and the wrap 44, the sensor can be configured to sense the actual compressive pressure at the interface between the patient's body, the sleeve 42, and the wrap 44. In either configuration—those in which the sensor is placed between the body and the sleeve 42 or those in which the sensor is placed between the sleeve 42 and the wrap 44—the sensor can sense changing pressure in the body area being monitored. In this way, the patient and/or caregiver can readily determine the actual amount of applied compressive pressure at any time and make adjustments as needed.

Embodiments of the body monitoring system 10 allow sensors to be positioned at various and multiple locations in the wearable device 40. For example, sensors can be positioned at the instep, ankle, calf, and other anatomical locations. As a result, real-time measurements of the variable(s) can be monitored simultaneously across the entire wearable device 40. Such flexibility in measurement allows the benefit of monitoring, for example, actual applied compressive pressures along a graduated compression device.

In addition, the compressive pressure sensor can be adapted to take measurements of applied compressive pressure at multiple points within a particular sensor field. For example, the knitted cuff sensor 24 or the knitted-in sensor circuit 20 having a horizontal configuration can take measurements of applied compressive pressure simultaneously at multiple points about a circumference of the limb 34 on which the device 32 is being worn. Averaged measurements of applied compressive pressure provide the advantage of increased accuracy over individual point measurements. Thus, such multiple point measurements of compressive pressure can be averaged to provide a more accurate representation of actual compressive pressure being applied across a defined area.

In some embodiments, variables measured by a sensor can be transmitted to a data display, processing, and/or recording device 18. Various mechanisms can be utilized to display, process, transmit, and/or record measurements of the sensed variable(s). In some embodiments, variable data can be transmitted from a point of measurement to a miniature microprocessor and display unit 18 attached to the wearable device 40. The miniature display unit 18 is preferably an electronic display unit 18, for example, a miniature LCD or LED display screen.

The electronic display unit 18 can be attached to the wearable device 40 in various ways and locations. In one embodiment, the display unit 18 can be attached to the wearable device 40 using a clamping mechanism. In another embodiment, the wearable device 40 can have knit at a desired location on the device the cuff 22 or pocket 26 for housing the display unit 18. For example, the pocket 26 can be knit at the top, or proximal end, of a compressive pressure stocking, for example. The display unit 18 can be placed inside the pocket 26 such that the display unit 18 does not touch the patient's body.

The electronic display unit 18 can display the amount of compressive pressure actually being applied in a particular sensing area at any given time. In this way, persons managing compressive pressure therapy can adjust the compressive pressure device 32 while attending the patient without having to review the data at another location. Alternatively, or in addition, such data can be transmitted wirelessly to a computer at another location. Recording transmitted compressive pressure data can beneficially provide a clinical record of compressive pressure therapy for a patient over time. Such data display, transmission, and/or recording mechanisms 18 can be utilized with any embodiment of a body monitoring system 10 according to the present invention.

Some embodiments of the body compression monitoring system 30 can include compression level alarms. For example, if actual compressive pressure falls below a set minimum threshold, the system can trigger a low pressure alarm. That is, if actual applied compressive pressure drops below a certain level due to decrease in edema underneath the compressive pressure device, fabric fatigue, or other reason, the system can send a signal (visual and/or auditory) to the local display unit 18 and/or to a remote location that the pressure is too low. The system 10 can also provide a high pressure alarm that similarly alarms when pressure becomes too high, such as when the device 32 slips out of position or edema increases.

Embodiments of the body monitoring system 10 and/or method provide a mechanism for accurately determining an actual amount of compressive pressure applied by a compressive pressure device to a patient. Such a body compression monitoring system 30 and/or method can provide accurate measurements of compressive pressure applied over the entire area or in selected areas underneath the compressive pressure device 32. Such a body compression monitoring system 30 and/or method can provide accurate measurements of applied compressive pressure the entire time the device is being worn.

In some embodiments, measurement and/or recording of the variable(s) can be continuous or at selected intervals. Such dynamic clinical information facilitates the administration of therapeutic amounts of compressive pressure, for example, so as to achieve desired outcomes. Accordingly, as a result of such accurate and ongoing information, systems and methods according to the present invention can facilitate optimized care in the treatment and prevention of vascular and other conditions.

In addition, documentation of actual applied compressive pressure can enhance risk management related to clinical practice, and can a record of treatment for reimbursement purposes.

Embodiments of the body compression monitoring system 30 and/or method can be easily utilized by clinicians, as well as by patients or other non-clinicians.

Embodiments of the body compression monitoring system 30 and/or method can be utilized in combination with other compression therapy devices. For example, the body compression monitoring system 30 can be utilized in combination with stockings, hosiery, sleeves, wraps, bandages, and/or other means for providing compression therapy. Some embodiments can be positioned adjacent a wearer's skin with another compression therapy garment overlying the body compression monitoring system 30. In other embodiments, the body compression monitoring system 30 can be applied over another compression therapy garment. In either case, the body compression monitoring system 30 can be utilized to accurately monitor compressive pressure actually applied by the combination of compression therapy means.

Embodiments of the body monitoring system 10 and/or method provide a mechanism for accurately measuring body variables regardless of variables related to yarn, fabric construction, stretch characteristics, number of fabric layers, yarn/fabric fatigue, body shape and circumference, and other variables related to a therapeutic wearable device (such as wearable device 40) and its application.

Some embodiments of such a body compression monitoring system 30 and/or method may be useful for allowing a user to easily and accurately determine compressive pressure at different locations on a person's body. In such a body compression monitoring system 30 and/or method, accurate measurements of applied compressive pressure at various anatomical locations, for example, along a leg, can provide assurance that compressive pressures are appropriately graduated.

Various embodiments of the body compression monitoring system 30 and/or method can be utilized on different anatomical areas. For example, some embodiments of the body compression monitoring system 30 and/or method can be utilized to monitor compressive pressure applied to a leg in treatment of venous insufficiency or a venous ulcer. Other embodiments can be utilized to monitor compressive pressure applied to an arm in treatment of lymphedema. Yet other embodiments can be utilized to monitor compressive pressure applied to a chest following breast surgery or to an abdomen after a liposuction procedure. The range within which actual applied compressive pressure may be accurately monitored can vary, depending on the amount of compression to be applied by a device. For example, the range of compressive pressure to be applied in treatment of lymphedema in an arm may be higher than the range of compressive pressure to be applied in treatment of venous insufficiency in a leg. Accordingly, the range within which actual applied compressive pressure may be accurately monitored in the lymphedema application would be greater than that for a venous insufficiency application.

Although the present invention has been described with reference to particular embodiments, it should be recognized that these embodiments are merely illustrative of the principles of the present invention. Those of ordinary skill in the art will appreciate that a body monitoring system and/or methods of the present invention may be constructed and implemented in other ways and embodiments. For example, embodiments of the body monitoring system 10 described herein refer to the compressive pressure device 32 that is knitted and to sensors, cuffs 22, pockets 26, and circuits 16, 20 that are knit into the device. It is understood that the inventive aspects of a body monitoring system 10 and/or methods of the present invention may be utilized in the compressive pressure device 32 that is woven and to sensors, cuffs 22, pockets 26, and circuits 16, 20 that are woven into the device 32. Accordingly, the description herein should not be read as limiting the present invention, as other embodiments also fall within the scope of the present invention.

What is claimed is:

1. A body monitoring system, comprising:
a wearable device; and
a circuit for conducting electrical signals comprising an electrically conductive yarn knitted into the device.

2. The system of claim 1, wherein the circuit further comprises a sensor circuit configured to sense a variable in an area of a body to which the device is applied.

3. The system of claim 1, wherein the circuit further comprises a transmission circuit configured to transmit an electrical signal representing a value of a variable in an area of a body to another location.

4. The system of claim 2, wherein the sensor circuit further comprises an electrical sensitivity for reliably sensing the variable.

5. The system of claim 3, wherein the transmission circuit further comprises an electrical sensitivity for reliably transmitting the value of a variable.

6. The system of claim 1, wherein the electrically conductive yarn comprises a silver yarn or a yarn coated with silver.

7. The system of claim 6, wherein the electrically conductive yarn is selected from a group consisting of a single 70 denier silver yarn and two 70 denier silver yarns twisted together.

8. The system of claim 1,
wherein the electrically conductive yarn comprises stitch loops, and
wherein the stitch loops are packed together during knitting so that the stitch loops in adjacent courses along a particular wale have sufficient contact to provide a continuous circuit.

9. The system of claim 1,
wherein the electrically conductive yarn comprises nylon yarn having silver or a silver composition applied thereto, and
wherein the nylon yarn is heated sufficiently to shrink the nylon yarn so that stitch loops in adjacent courses along a particular wale have sufficient contact to provide a continuous circuit.

10. The system of claim 1, wherein the circuit further comprises the electrically conductive yarn knit in a vertical, horizontal, or angled direction in the fabric.

11. The system of claim 10, wherein the electrically conductive yarn comprises a knit rib pattern to provide a vertical circuit direction in the fabric.

12. The system of claim 10, wherein the electrically conductive yarn is knit along a course to provide a horizontal circuit direction in the fabric.

13. The system of claim 10, wherein the electrically conductive yarn is knit in a wale offset from a previous wale in successive courses to provide an angled circuit direction in the fabric.

14. The system of claim 10, wherein the electrically conductive yarn is laid in
(a) a single course to provide a horizontal circuit direction,
(b) a plurality of courses to provide an angled circuit direction, or
(c) changing directions between courses to provide a multi-directional circuit direction.

15. The system of claim 1, the wearable device comprising an elastic fabric having an unstretched dimension in a direction of the circuit, wherein stretch beyond the unstretched dimension in the circuit direction is limited to provide sufficient circuit continuity for reliable conduction of the electrical signals.

16. The system of claim 15,
wherein the circuit further comprises a cut yarn, and
wherein stretch is limited to about 5-10% beyond the unstretched dimension in the circuit direction.

17. The system of claim 15,
wherein the circuit further comprises a continuously knit stretch nylon yarn, and
wherein stretch is limited to about 10-20% beyond the unstretched dimension in the circuit direction.

18. The system of claim 15,
wherein the circuit further comprises a continuously knit 70 denier spandex yarn, single or double covered with a conductive nylon yarn, and
wherein stretch is limited to about 50-100% beyond the unstretched dimension in the circuit direction.

19. The system of claim 3, wherein the another location to which the electrical signal is transmitted comprises an external device separate from the wearable device.

20. The system of claim 19, wherein the external device comprises an electronic display unit configured to display the transmitted value of a variable.

21. The system of claim 1, wherein the circuit is configured to conduct electrical signals in both directions along the circuit.

22. The system of claim 1, wherein the circuit is configured to transmit power from a power source to a location on the wearable device.

23. The system of claim 2, wherein the wearable device comprises a compressive pressure device, and wherein the variable comprises compressive pressure applied by the device.

24. The system of claim 3,
wherein the wearable device comprises a compressive pressure device and a sensor configured to sense compressive pressure in an area of a body to which the device is applied, and
wherein the transmission circuit is configured to transmit an electrical signal representing an amount of compressive pressure sensed in the area of a body to an external electronic display unit.

25. The system of claim 24,
wherein the compressive pressure device comprises an inner compressive pressure sleeve and an overlying outer compressive pressure wrap,
wherein the sensor is located either (a) between the body and the sleeve, (b) within the sleeve (c) between the sleeve and the wrap, or (d) within the wrap, and
wherein the sensor is configured to sense an actual cumulative amount of compressive pressure applied by the sleeve and the wrap.

26. A body monitoring system, comprising:
a wearable device comprising an elastic fabric; and
a circuit for conducting electrical signals comprising an electrically conductive silver yarn or a yarn coated with silver knitted into the device fabric in a vertical, horizontal, or angled direction, the circuit further comprising
(a) a sensor circuit configured to sense a variable in an area of a body to which the device is applied, and
(b) a transmission circuit configured to transmit an electrical signal representing a value of the variable in the area of the body to an external electronic display unit configured to display the transmitted value of the variable,
wherein the device fabric has an unstretched dimension in a direction of the circuit, and
wherein stretch beyond the unstretched dimension in the circuit direction is limited to provide sufficient circuit continuity for reliable conduction of the electrical signals.

27. The system of claim 26,
wherein the wearable device comprises a compressive pressure device,
wherein the variable comprises compressive pressure applied by the device, and
wherein the transmission circuit is configured to transmit an electrical signal representing an amount of compressive pressure sensed in the area of a body to an external electronic display unit.

28. A body monitoring system, comprising:
a wearable device;
a sensor configured to sense a variable in an area of a body to which the device is applied; and
a transmission circuit comprising an electrically conductive yarn knitted into the device and configured to transmit an electrical signal representing a value of the variable in the area of a body to another location.

29. The system of claim 28, wherein the sensor further comprises a knitted cuff sensor.

30. The system of claim 29, wherein the knitted cuff sensor comprises a three-layer capacitance type sensor comprising
(a) an inner layer electrically conductive yarn,
(b) a middle layer semi-conductive dielectric yarn, and
(c) an outer layer electrically conductive yarn.

31. The system of claim 29, wherein the knitted cuff sensor comprises a two-layer capacitance type sensor comprising
(a) an inner cuff layer and an outer cuff layer each comprising an electrically conductive yarn and
(b) an electrically regulating dielectric material inserted between the inner and outer cuff layers.

32. The system of claim 29, wherein the knitted cuff sensor comprises a piezoelectric type sensor.

33. The system of claim 29, wherein the knitted cuff sensor comprises a piezoresistive sensor comprising
(a) an inner cuff layer and an outer cuff layer each comprising an electrically conductive silver yarn and
(b) a piezoresistive semi-conductive polymer disposed between the inner and outer cuff layers.

34. The system of claim 28, further comprising a cuff integrally knit into the wearable device,
wherein the cuff is configured to house the sensor, and
wherein the sensor comprises an electro-mechanical sensor, a capacitance sensor, or a piezoelectric sensor.

35. The system of claim 28, further comprising a pocket integrally knit into the wearable device,
wherein the pocket is configured to house the sensor, and
wherein the sensor comprises an electro-mechanical sensor, a capacitance sensor, or a piezoelectric sensor.

36. The system of claim 28,
wherein the sensor is securable to a hook-and-loop type fastener engagable with the wearable device, and
wherein the sensor comprises an electro-mechanical sensor, a capacitance sensor, or a piezoelectric sensor.

37. The system of claim 28, wherein the sensor further comprises a sensor circuit printed onto a material comprising a hook-and-loop type fastener engagable with the wearable device.

38. The system of claim 37, further comprising an electrically conductive yarn sewn through the material so that the yarn is conductively contactable between the printed sensor circuit and the transmission circuit in the wearable fabric.

39. The system of claim 28,
wherein the wearable device comprises a compressive pressure device,
wherein the variable comprises compressive pressure,
the system further comprising a pressurized cuff (a) having opposing ends releasably securable to each other, (b) adjustably positionable about the wearable device, and (c) having the sensor integrated into the cuff,
wherein when the pressurized cuff is adjusted about the wearable device to have the same initial compressive pressure as the wearable device, the sensor senses changes in actual applied pressure at an interface of the body area, the wearable device, and the pressurized cuff.
